(19) United States
(12) Patent Application Publication
(10) Pub. No.: US 2003/0100594 A1
(43) Pub. Date: May 29, 2003

(54) Carbonic anhydrase inhibitor

(75) Inventors: Jaime L. Masferrer, Ballwin, MO (US); Janet M. O'Neal, Richmond Heights, MO (US)

Correspondence Address: POLSTER, LIEDER, WOODRUFF & LUCCHESI, 763 SOUTH NEW BALLAS ROAD, ST. LOUIS, MO, 63141-8750, US

(73) Assignee: Pharmacia Corporation

(21) Appl. No.: 10/213,793

(22) Filed: Aug. 7, 2002

Related U.S. Application Data
(60) Provisional application No. 60/311,561, filed on Aug. 10, 2001.

(51) Int. Cl. A61K 31/415

(52) U.S. Cl. 514/406

(57) ABSTRACT

Methods of treating or preventing carbonic anhydrase associated disorders or diseases in a subject in need of such treatment as prevention comprising the administration of cyclooxygenase-2 inhibitors, or structurally related compounds, having carbonic anhydrase inhibitory properties, as well as combinations of the cyclooxygenase-2 inhibitors, or structurally related compounds, with anti-inflammatory agents or drugs, antineoplastic agents or drugs or ophthalmic agents or drugs in methods of treatment and prevention of disorders or diseases.

### CROSS REFERENCE TO RELATED APPLICATIONS

This application claims priority to provisional application Serial No. 60/311,561 filed Aug. 10, 2001.

### STATEMENT REGARDING FEDERALLY SPONSORED RESEARCH OR DEVELOPMENT

Not applicable

### FIELD OF INVENTION

The present invention relates in general to the use of carbonic anhydrase inhibitors, drugs, or agents in medicine and, more specifically, to the use of compounds that exhibit carbonic anhydrase (CA) inhibitor activity and compounds that exhibit both carbonic anhydrase (CA) and cylcooxygenase-2 (COX-2) inhibitor activity, in methods of treatment of diseases associated with the isozymes of carbonic anhydrase or with COX-2, or both.

### BACKGROUND OF THE INVENTION

Carbonic anhydrases are metalloprotein enzymes which catalyze the hydration of carbon dioxide and the dehydration of bicarbonate: CO₂+H₂O→HCO⁻₃+H⁺. The carbonic anhydrases are widespread in nature and found in animals, plants and certain bacteria. In humans CA has at least fourteen (14) isoenzymes with different physiological functions. (Scozzafava et al, J. Med. Chem., 43:3677-3687, 2000). The CA isozymes are involved in respiration and transport of CO₂/bicarbonate between metabolizing tissues and the lungs, pH homeostasis, electrolyte secretion in a variety of tissues, and biosynthetic reactions such as lipogenesis, gluconeogenesis, and ureagenesis.

Carbonic anhydrase inhibitors initially were developed as diuretics for the treatment of edema. One mechanism of the diuretic action is due to the inhibitory effect of sulfanilamide on the carbonic anhydrase enzyme, resulting in increased bicarbonate excretion and obligatory water loss through the kidneys. Although carbonic anhydrase inhibitors may be used to treat edema associated with congestive heart failure and for drug-induced edema, presently the major indication for carbonic anydrase inhibitors is for treatment of open-angle glaucoma. Also the carbonic anhydrase inhibitors may be used to treat secondary glaucoma and preoperatively in acute angle closure glaucoma before surgery.

Carbonic anhydrase inhibitors also are used to treat optic neuropathy associated with elevated intracranial pressure and to treat pseudomotor cerebri in headache management. Carbonic anhydrase inhibitors have been used to treat cystoid macular edema (CME). (Wolfensberger, T. J., Doc Opthalmol 1999; 97 (3-4):387-97).

Acetazolamide has been shown to potentiate the antitumor activity of 1-phthalidyl 5-fluorouracil (PH-5-FU). (Hisanori Kaisai. et. al, Cancer Chemother Pharmacol. 1986; 16(l):55-7). Acetazolamide was shown to reduce invasiveness of certain renal cancer cell lines. (Parkkila, S. et al, PNAS, 95:5:2220-2224, 2000). Sulfonamide carbonic anhydrase inhibitors have been shown to inhibit cell growth in leukemia, non-small cell lung cancer, ovarian cancer, melanoma, colon, CNS, renal, prostate and breast cancer cell lines. (C. Supuran, et al, Eur. J. Med. Chem, 35: 867-874 (2000)).

### SUMMARY OF THE INVENTION

One exemplary embodiment of the invention provides a method of treating or preventing a carbonic anhydrase-associated disorder in a subject in need of such treatment or prevention comprising the administration to the subject a carbonic anhydrase inhibitor treating- or preventing- effective amount of a cyclooxygenase-2 inhibitor, a pharmaceutically acceptable salt thereof or prodrug to treat or prevent the disorder.

Another exemplary embodiment of the invention provides a method of treating or preventing carbonic anhydrase-associated disorders in a subject in need of such treatment or prevention comprising the administration to the subject a carbonic anhydrase inhibitor treating- or preventing-effective amount of a selective cyclooxygenase-2 inhibitor a pharmaceutically acceptable salt thereof or prodrug to treat or prevent the disorder.

One exemplary embodiment of the invention provides a method of treating or preventing a carbonic anhydrase associated disorder in a subject in need of such treatment or prevention comprising the administration to the subject a carbonic anhydrase inhibitor treating- or preventing-effective amount of a cyclooxygenase-2 inhibitor compound having a sulfonamide structure thereon, a pharmaceutically acceptable salt thereof or prodrug to treat or prevent the disorder.

Another exemplary embodiment of the invention provides a method of treatment of a neoplastic disorder or disease in a subject in need of such treatment or prevention comprising the administration to the subject a carbonic anhydrase inhibitor treating- or preventing-effective amount of a cyclooxygenase-2 inhibitor a pharmaceutically acceptable salt thereof or prodrug to treat or prevent the disorder.

Another exemplary embodiment of the invention provides a method of treatment of a neoplastic disorder or disease in a subject in need of such treatment or prevention comprising the administration to the subject a carbonic anhydrase inhibitor treating- or preventing-effective amount of a cyclooxygenase-2 inhibitor a pharmaceutically acceptable salt thereof or prodrug to treat or prevent the disorder wherein the neoplastic disorder includes, but is not limited to renal cancer, leukemia, lung cancer, ovarian cancer melanoma, colon cancer, cancer of the central nervous system, prostate cancer and breast cancer.

Another exemplary embodiment of the invention provides a method of treating or preventing carbonic anhydrase -associated disorders in a subject in need of such treatment or prevention comprising the administration to the subject a carbonic anhydrase inhibitor treating- or preventing-effective amount of a cyclooxygenase-2 inhibitor a pharmaceutically acceptable salt thereof or prodrug to treat or prevent the disorder wherein the carbonic anhydrase-associated disorder includes, but is not limited to, edema, open-angle glaucoma, secondary glaucoma, acute angle closure glaucoma, epilepsy, acute mount sickness, familial periodic paralysis, metabolic alkylosis, optic neuropathy, pseudomotor cerebri, and cystoid macular edema.

An exemplary embodiment of the invention provides a method of treatment of a neoplastic disorder or disease in a subject in need of such treatment or prevention comprising the administration to the subject an antineoplastic drug or agent and a carbonic anhydrase inhibitor treating- or preventing-effective amount of a cyclooxygenase-2 inhibitor a pharmaceutically acceptable salt thereof or prodrug to prevent or treat the neoplastic disorder.

One exemplary embodiment of the invention provides a method of treating or preventing a carbonic anhydrase associated disorder in a subject in need of such treatment or prevention comprising the administration to the subject a carbonic anhydrase inhibitor treating- or preventing-effective amount of a compound selected from the group of compounds of the formulas

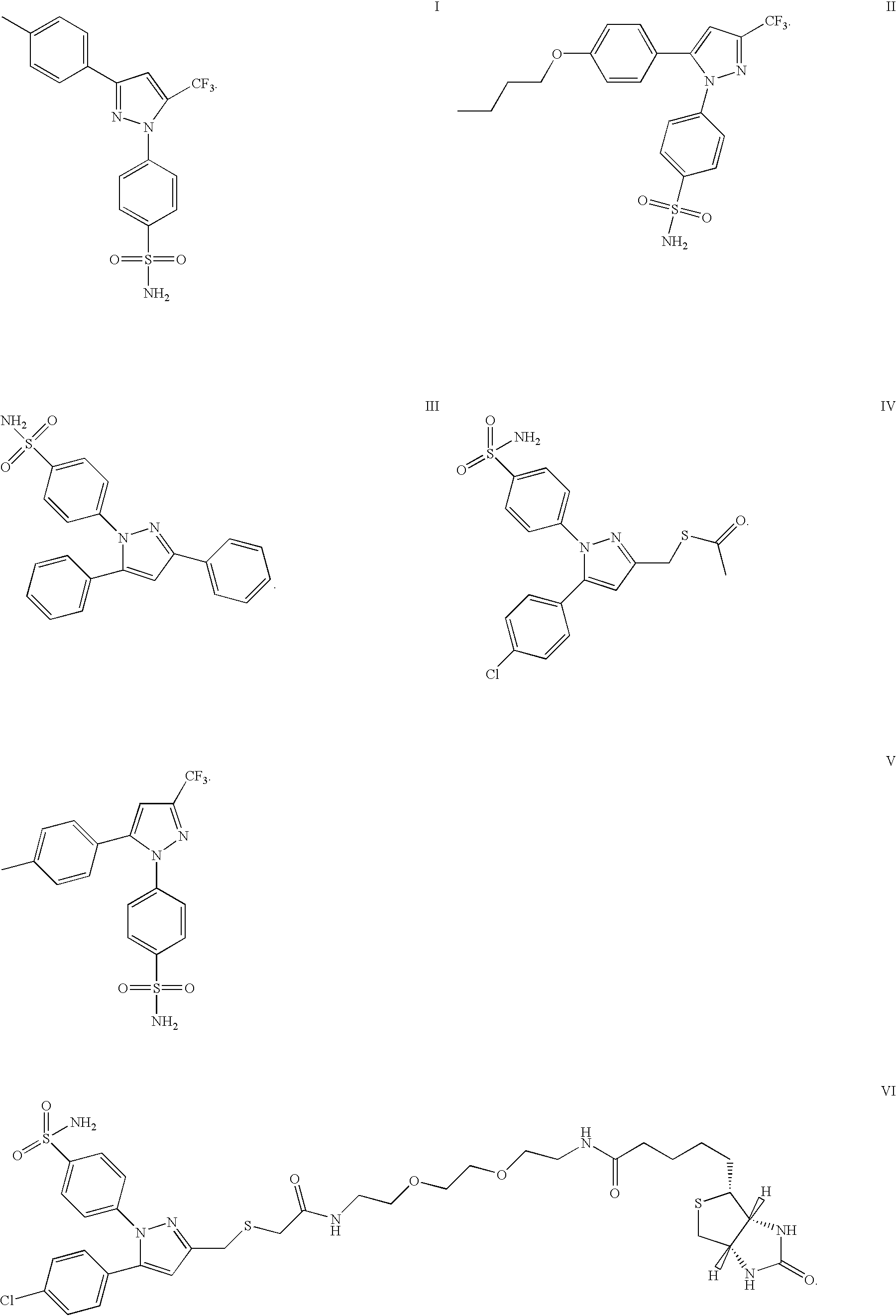

or a pharmaceutically acceptable salt thereof or prodrug.

One exemplary embodiment of the invention provides a method of treating or preventing a carbonic anhydrase associated disorder in a subject in need of such treatment or prevention comprising the administration to the subject a carbonic anhydrase inhibitor treating- or preventing-effective amount of a selective cyclooxygenase-2 inhibitor selected from the group consisting of the formulas

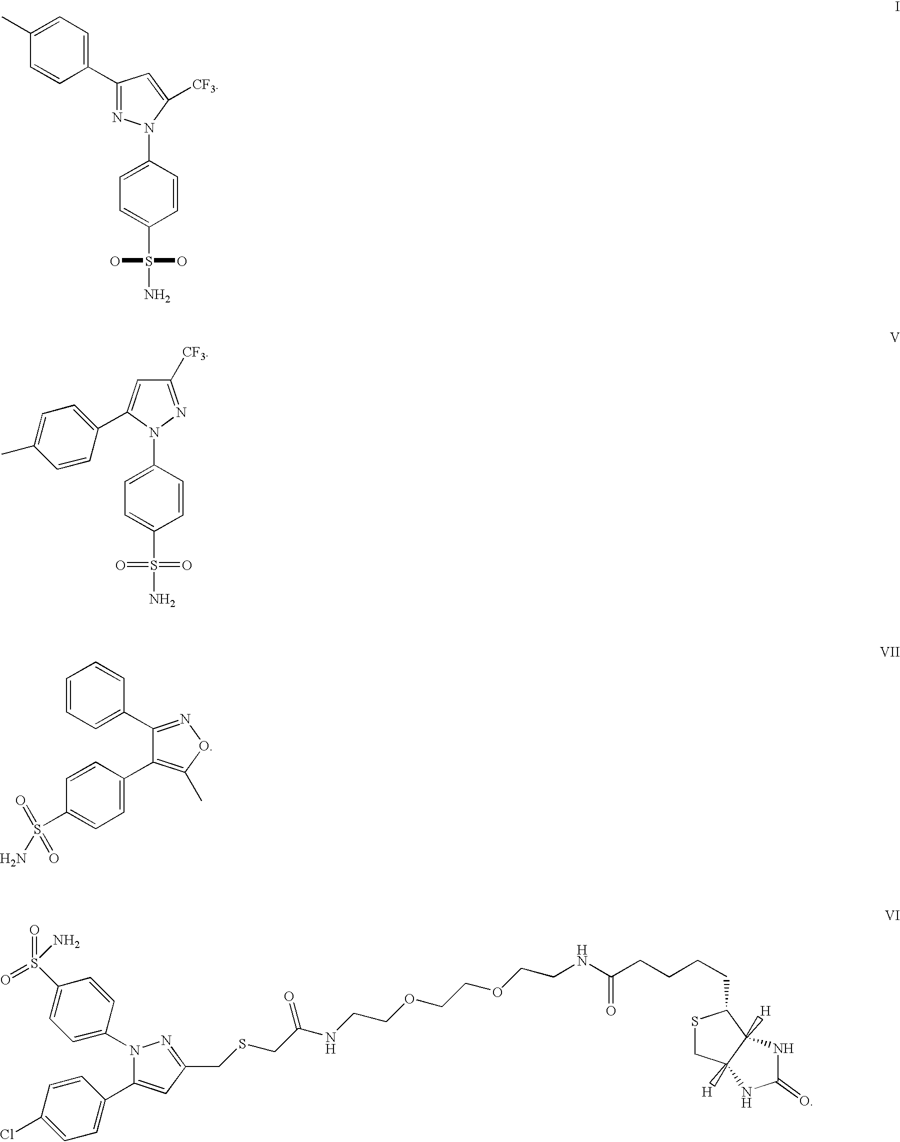

or a pharmaceutically acceptable salt thereof or prodrug.

Another aspect of the invention includes a method of prevention or treating an ophthalmic disorder or disease in a subject in need of such prevention or disease comprising the administration of a ophthalmic disorder or disease preventing or treating amount of a an ophthalmologic agent or drug and a carbonic anhydrase inhibitor selected from the group of compounds consisting of the compound of formulas

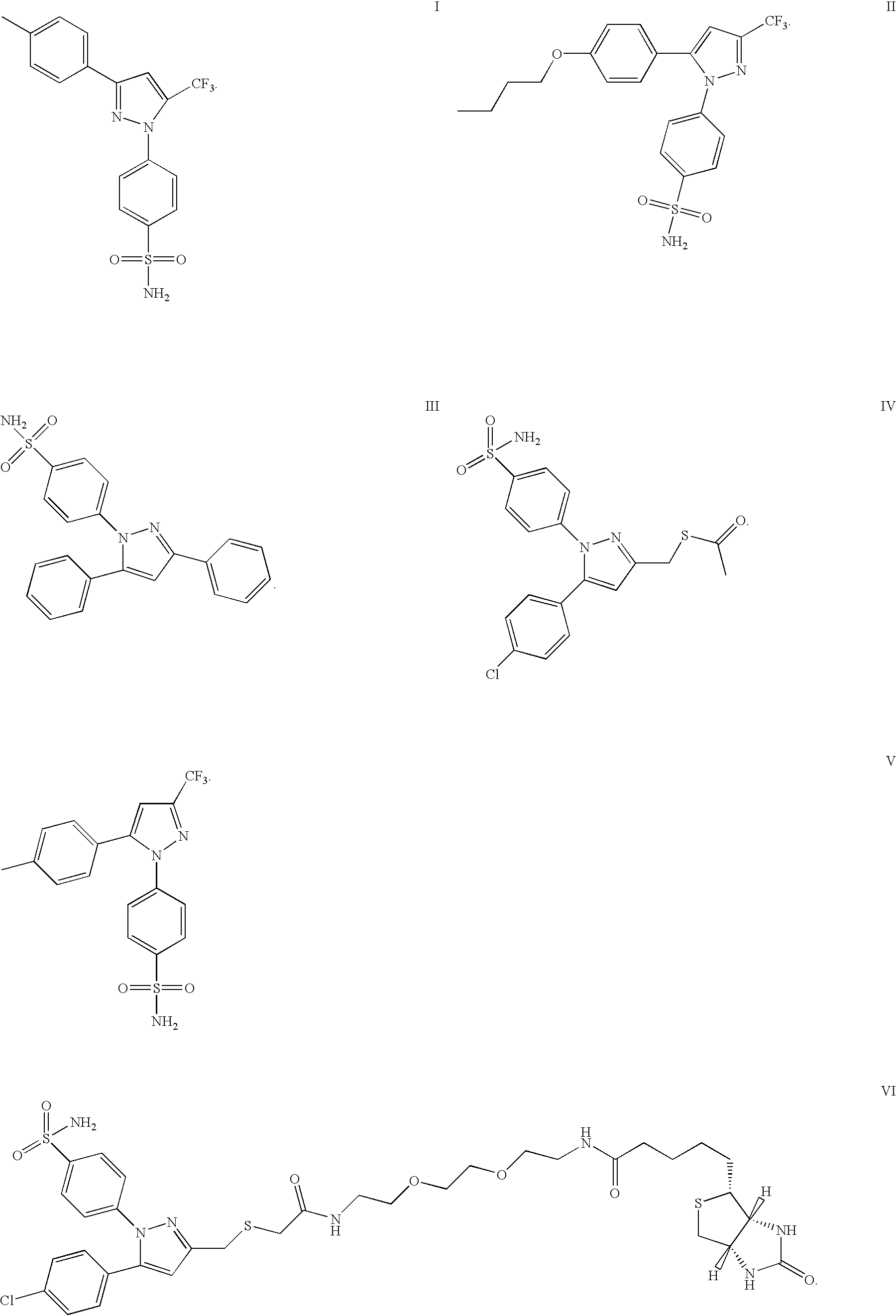

or a pharmaceutically acceptable salt thereof or prodrug.

The invention also includes a method for treating or preventing a neoplasia disorder in a mammal in need of such treatment or prevention, which method comprises treating the mammal with a therapeutically effective amount of a combination comprising an antineoplastic drug or agent and a carbonic anhydrase inhibitor selected from the group of carbonic anhydrase inhibitors consisting of the formulas

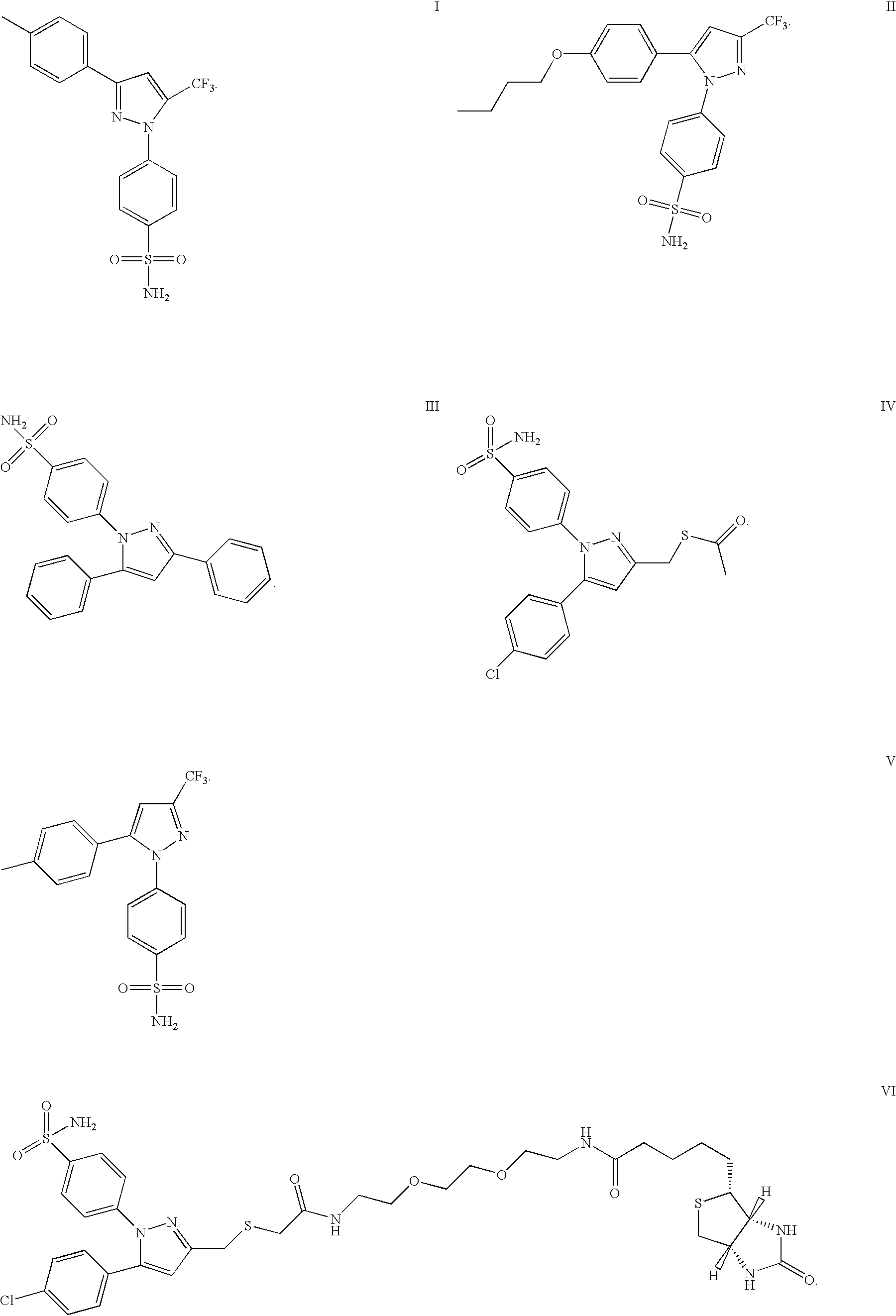

or a pharmaceutically acceptable salt thereof or prodrug.

### DETAILED DESCRIPTION OF THE INVENTION

Generally, the present invention encompasses agents that inhibit isozymes of carbonic anhydrase and their method of use in medicine in preventing and treating various diseases or conditions in which carbonic anhydrase is implicated or involved in metabolic pathways that influence the diseases or conditions. The term “carbonic anhydrases” as used herein refers to the metalloprotein enzymes which catalyze the simple interconversion of CO₂ and H₂CO₃ (CO₂+O₂→HCO₂⁻+H⁺). The term “carbonic anhydrase inhibitor” as used herein refers to agents that reduce or inhibit the activity of human carbonic anhydrases.

The invention also encompasses agents that exhibit more than one therapeutic effect in that they inhibit carbonic anhydrases and inhibit cyclooxygenase-2 (COX-2), concomitantly. The agents have utility in the treatment of carbonic anhydrase and COX-2 associated disorders, diseases or physiological conditions. The agents have therapeutic applications such as treatment of ophthalmic or ocular diseases such as glaucoma and macular degeneration, inflammatory conditions and neoplastic diseases or conditions. The invention also encompasses therapeutic combinations of the carbonic anhydrase inhibitors with other therapeutic agents such as ophthalmic drugs or agents and antineoplastic agents.

As set out in detail immediately below, Compounds I, II, III, IV, V, VI and VII demonstrate carbonic anhydrase inhibition in vitro. In summary, Compound I is a potent inhibitor of carbonic anhydrase with a IC₅₀ of 20 nM. Compound I is a more potent inhibitor than acetazolamide (IC₅₀ of 30 nM). The selective COX-2 inhibitors having sulfonamide structures, celecoxib (Compound V) and valdecoxib (Compound VII), inhibit carbonic anhydrase with an average IC₅₀ of 140 nM and 330, respectively. The selective COX-2 inhibitor rofecoxib (Compound VIII) did not exhibit significant carbonic anhydrase inhibition.

### EXAMPLE

Objective:

To investigate the inhibitory activity of COX-2 inhibitors and other structurally related compounds on human carbonic anhydrase II activity.

Compounds Tested:

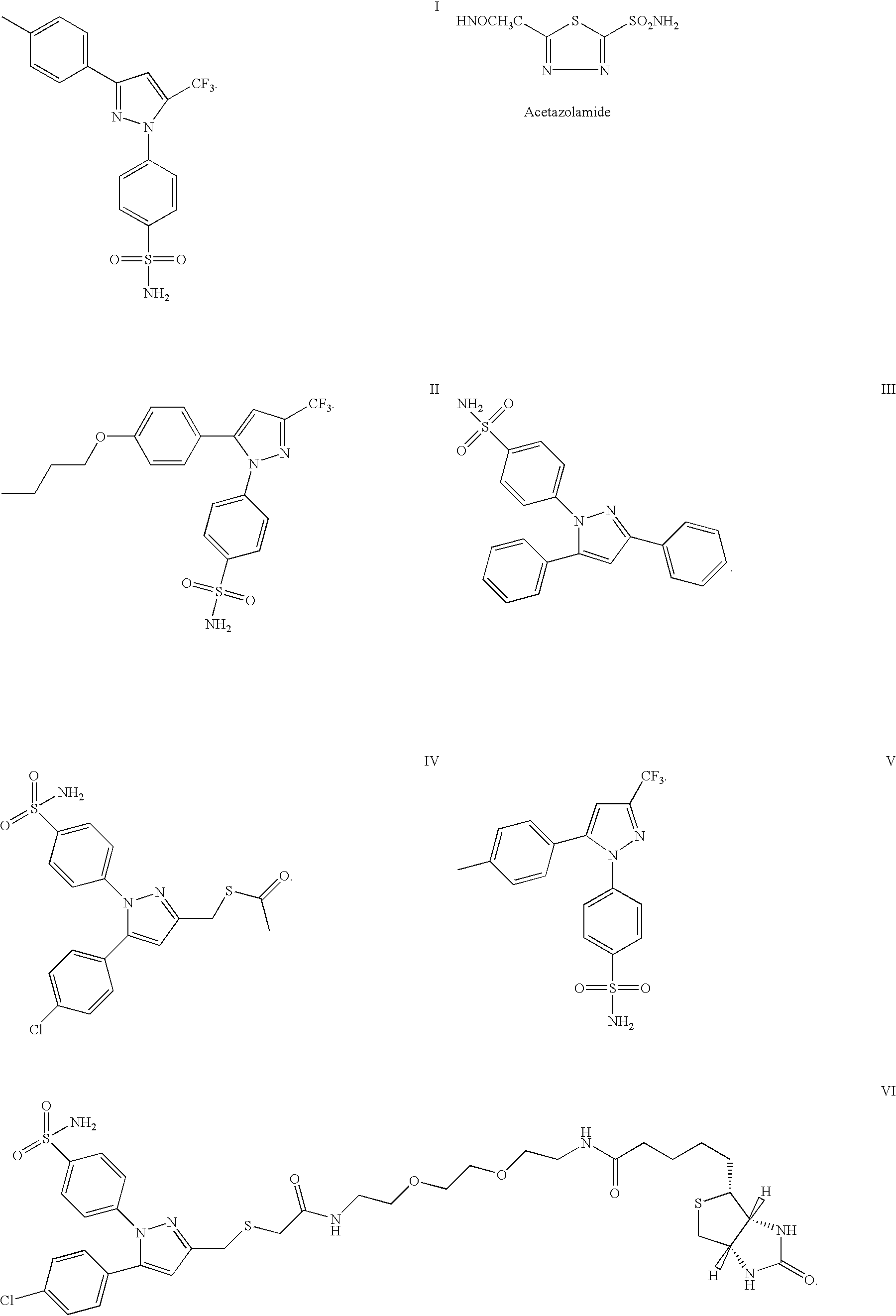

Description of the Compounds:

Acetazolamide (5-Acetamido-1,3,4-thiadiazole-2-sulfonamide) is a known carbonic anhydrase inhibitor included in the study as a standard.

Compounds I, II, III, IV, V, VI, VII, VIII, IV and X are structurally related compounds represented by the general structure of Formula A.:

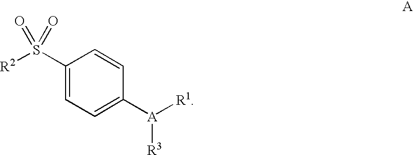

wherein A is selected from the group consisting of partially unsaturated or unsaturated heterocyclyl and partially unsaturated or unsaturated carbocyclic rings;

wherein R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

wherein R² is selected from the group consisting of methyl or amino; and

wherein R³ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; or a pharmaceutically acceptable salt thereof.

Compound V (celecoxib) is a selective cyclooxygenase-2 inhibitor, described in detail in U.S. Pat. No. 5,466,823, which is incorporated herein by reference. Compound VII (valdecoxib) also is a selective cyclooxygenase-2 inhibitor, disclosed in detail in U.S. Pat. No. 5,633,272, incorporated herein by reference. Compound VIII (rofecoxib) is a selective cyclooxygenase-2 inhibitor, described in detail in U.S. Pat. No. 5,691,375, which is incorporated by reference. Compound IV exhibits cyclooxygenase inhibitor activity, but appears to be more selective for COX-2 than COX-1. Compound I and Compound VI do not inhibit cyclooxygenase-1 (COX-1) but weakly inhibit COX-2. Compounds II, III, IX, and X do not exhibit cyclooxygenase inhibitory activity.

The terms “cyclooxygenase-1” and “COX-1” used interchangeably herein refer to the constitutive isoform of the enzyme cyclooxygenase. The terms “cyclooxygenase-2” and “COX-2 as used interchangeably herein refer to the inducible isoform of the enzyme cyclooxygenase. The term “COX-2 selectivity” has been given numerous and varied definitions in the published literature. Selectivity has been understood to refer, alternatively, to a variety of in vitro conditions and to a variety of in vivo conditions. In vitro selectivity does not necessarily mean the same thing as in vivo selectivity. However, as used herein, the terms “cyclooxygenase-2 selective inhibitor” and “COX-2 selective inhibitor” are used interchangeably herein and for the present invention refer to a therapeutic compound that inhibits cyclooxygenase-2 more than it inhibits cyclooxygenase-1 in an in vitro recombinant enzyme assay. The term “cyclooxygenase-2 inhibitor” or “COX-2 inhibitor” refers to any compound which inhibits the COX-2 enzyme, without regard to the extent to which it inhibits COX-1. Especially suitable as cyclooxygenase-2 selective inhibitors useful in the present invention are those compounds that have a cyclooxygenase-2 IC₅₀ of less than about 0.2 μM, and also have a selectivity ratio of cyclooxygenase-2 inhibition over cyclooxygenase-1 inhibition of at least 50, and more preferably of at least 100. Even more preferably, the cyclooxygenase-2 selective inhibitor compounds have a cyclooxygenase-1 IC₅₀ of greater than about 1 μM, and more preferably of greater than 10 μM.

Materials and Methods:

| | |
|---|---|
| 100 μL | 0.04 M Tris Buffer pH 7.6 |
| 10 μL | Carbonic Anhydrase II Enzyme 500 Units/mL (Sigma C-6165) |
| 20 μL | Inhibitor Compound |
| 70 μL | 3mM p-nitrophenyl acetate substrate (Sigma N-8130) |

Incubate at room temperature in 96 well plate and read absorbance at 405 nm.

Table 1, below, lists the assay results for the compounds:

TABLE 1

| | CA Assay | SULFONAMIDE |
|---|---|---|
| COMPOUND | N IC50 (μM) | STRUCTURE? |
| I | 3 0.01 (0.015, 0.021, .004) | YES |
| Acetazolamide | 4 0.03 (0.04, 0.017, 0.03, | YES |
| | .017) | |
| II. | 2 0.04 (0.03, 0.04) | YES |
| III. | 1 0.04 | YES |
| IV | 1 0.09 | YES |
| V. | 3 0.14 (0.16, 0.15, .10) | YES |
| Celecoxib | | |
| VI. | 1 0.18 | YES |
| VII. | 2 0.33 (0.4, 0.25) | YES |
| Valdecoxib | | |
| VIII. | 1 >100 | NO |
| Rofecoxib/Vioxx | | |
| IX. | 1 >100 | NO |
| X. | 1 >100 | NO |

Results:

All compounds tested containing a sulfonamide structure inhibited CA II. The selective COX-2 inhibitors, celecoxib and valdecoxib, inhibited CA II activity with IC50s of 0.14 μM and 0.33 μM, respectively. The selective COX-2 inhibitor rofecoxib did not inhibit the enzyme up to 100 μM. The known inhibitor of carbonic anhydrase, acetazolamide, and Compound I, blocked CA II activity with IC50s of 0.03 μM and 0.01 μM, respectively.

Methods of Treatment:

The compounds shown to inhibit carbonic anhydrase can be used in methods of treatment or prevention of any carbonic anhydrase associated disorder, disease or physiological condition in a subject in which the inhibition of carbonic anhydrase enzymes effects treatment or prevention of the disorder, disease or physiological condition. The Compounds I, II, III, IV, V, VI, and VII, or pharmaceutical salts thereof or prodrugs may be used for any medical indication in which carbonic anhydrase inhibitors have been shown to be effective or may be effective, alone or in combination. Furthermore, other related compounds having a sulfonamide group, and which exhibit carbonic anhydrase inhibition, are within the scope of the invention. For example, the following is an exemplary list of structurally related compounds known to be selective COX-2 inhibitors that include a sulfonamide group: Compound XI (deracoxib) and Compound XII (JTE-522) or a pharmaceutically acceptable salts or prodrug thereof.

TABLE 2
Examples of Other Tricyclic COX-2 Selective
Having A Sulfonamide Group

| Compound | |
|---|---|
| Number | Structural Formula |
| XI | 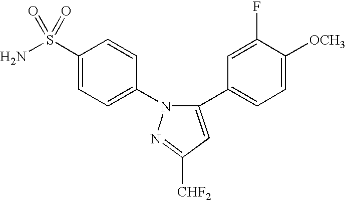 |
| XII | 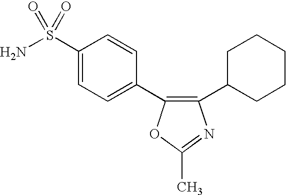 |

Compound XIII (parecoxib), below, which is a therapeutically effective prodrug of the tricyclic cyclooxygenase-2 selective inhibitor Compound VII (valdecoxib) (U.S. Pat. No. 5,932,598, herein incorporated by reference), may be advantageously employed as a source of a cyclooxygenase inhibitor having carbonic anhydrase inhibitor activity

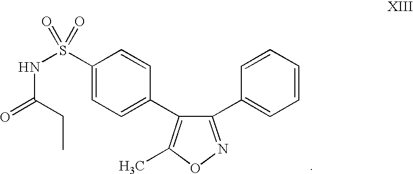

Suitable routes of administration of the compounds of the present invention include any means that produce contact of these compounds with their site of action in the subject's body. More specifically, suitable routes of administration include oral, intravenous, subcutaneous, rectal, topical, buccal (i.e. sublingual), intramuscular, and intradermal. In an exemplary embodiment, the combinations are orally administered.

Pharmaceutically acceptable salts are particularly suitable for medical applications because of their greater solubility relative to the parent compound. Such salts must clearly have a pharmaceutically acceptable anion or cation. Suitable pharmaceutically acceptable acid addition salts of the compounds of the present invention include when possible include those derived from inorganic acids, such as hydrochloric, hydrobromic, phophoric, metaphosphoric, nitric, sulfonic, sulfuric acids, and organic acids such as acetic, benzenesulfonic, benzoic, citric, ethanesulfonic, fumaric, gluconic, glycolic, isotho9nic, lactic, lactobionic, maleic, malic, methanesulfonic, succinic, toluenesulfonic, tartaric, and trifluoroacetic acids. The chloride salt is especially suitable for medical purposes. Suitable pharmaceutically acceptable base salts include ammonium salts, alkali metal salts such as sodium and potassium salts, and alkaline earth salts such as magnesium and calcium salts.

The compounds useful in the present invention are presented with an acceptable carrier in the form of a pharmaceutical combination. The carrier must be acceptable in the sense of being compatible with the other ingredients of the pharmaceutical combination and must not be deleterious to the subject. Suitable forms for the carrier include solid or liquid or both, and in an exemplary embodiment the carrier is formulated with the therapeutic compound as a unit-dose combination, for example as a tablet that contains from about 0.05% to about 95% by weight of the active compound. In alternative embodiments, other pharmacologically active substances are also present, including other compounds of the present invention. The pharmaceutical combinations of the present invention are prepared by any of the well-known techniques of pharmacy, consisting essentially of admixing the ingredients.

Preferred unit dosage formulations are those containing an effective dose, as herein below described, or an appropriate fraction thereof, of one or more of the therapeutic compounds of the combinations.

In general, a total daily dose of a cyclooxygenase-2 inhibitor in the combinations is in the range of about 0.3 to about 100 mg/kg body weight/day, preferably from about 1 mg to about 50 mg/kg body weight/day, and more preferably from about 3 mg to about 10 mg/kg body weight/day.

In the case of pharmaceutically acceptable salts of the therapeutic compounds, the weights indicated above refer to the weight of the acid equivalent or the base equivalent of the therapeutic compound derived from the salt.

It should be understood that the amount of each compound that is required to achieve the desired biological effect depends on a number of factors such as the specific individual compounds chosen, the specific use for which it is intended, the route of administration, the clinical condition of the subject, and the age, weight, gender, and diet of the subject.

The daily doses described in the preceding paragraphs for the various therapeutic compounds are administered in a single dose, or in proportionate multiple subdoses. Subdoses are administered from two to six times per day. In one embodiment, doses are administered in sustained release form effective to obtain the desired biological effect.

Oral delivery of the compounds of the present invention can include formulations, as are well known in the art, to provide prolonged or sustained delivery of the drug to the gastrointestinal tract by any number of mechanisms. These include, but are not limited to, pH sensitive release from the dosage form based on the changing pH of the small intestine, slow erosion of a tablet or capsule, retention in the stomach based on physical properties of the formulation, bioadhesion of the dosage form to the mucosal lining of the intestinal tract, or enzymatic release of the active drug from the dosage form.

Oral delivery of the compounds of the present invention can be achieved using a solid, semi-solid or liquid dosage form. Suitable semi-solid and liquid forms include, for example, a syrup or liquid contained in a gel capsule.

Pharmaceutical compositions suitable for oral administration can be presented in discrete units, such as capsules, cachets, lozenges, or tablets, each containing a predetermined amount of at least one of the therapeutic compounds useful in the combinations of the present invention; as a powder or in granules; as a solution or a suspension in an aqueous or non-aqueous liquid; or as an oil-in-water or water-in-oil emulsion.

One embodiment of the present invention is the treatment and prevention of carbonic anhydrases associated disorders or diseases in a subject in wherein administration of carbon anhydrase inhibitor to the subject is known to be effective in the treatment or prevention of the disorder or disease. These disorders and diseases include, but are not limited to, edema associated with congestive heart failure and for drug-induced edema; open-angle glaucoma, secondary glaucoma and preoperatively in acute angle closure glaucoma before surgery, epilepsy, the prophylaxis and symptomatic treatment of acute mountain sickness, familial periodic paralysis, metabolic alkalosis, particularly alkalosis caused by diuretic induced increases in H⁺ excretion, optic neuropathy associated with elevated intracranial pressure, pseudomotor cerebri in headache management, cystoid macular edema; cystoid macular edema due to retinitis pigmentosa.

Another embodiment of the invention is the treatment and prevention of neoplastic disorders or diseases in a subject wherein administration of carbon anhydrase inhibitor to the subject is effective in the treatment or prevention of the neoplastic disorder or disease. Such neoplastic disorders or diseases include, but not limited to, renal cancer, leukemia, non-small cell lung cancer, ovarian cancer, melanoma, colon cancer, CNS cancers, prostate and breast cancer.

One embodiment of the invention includes methods of treatment and prevention of carbonic anhydrases associated disorders or diseases in a subject in wherein administration Compounds I, II, III, IV, V, VI, or VI pharmaceutically effective salts thereof or prodrugs, to the subject is effective in the treatment or prevention of the disorder or disease.

Compound V (celecoxib) and Compound VII (valdecoxib), which are shown to inhibit carbonic anhydrase, are selective COX-2 inhibitors. Compound V and Compound VII, as well as other COX-2 inhibitors structurally related to Compound V and VII that have sulfonamide structures thereon, pharmaceutical salts or prodrugs thereof, may be used for any indications in which CA inhibitor and a COX-2 inhibitor would be indicated. Such indications include, but are not limited to, treating ophthalmic or ocular inflammation and more preferably in method of treatment of ophthalmic diseases such as retinitis, conjunctivitis, retinopathies, uveitis and ophthalmic or ocular photophobia, and of acute injury to eye tissue where there is increased intraocular pressure that responds to treatment with carbonic anhydrase inhibitor drugs or agents. Further, those compounds that are both COX-2 inhibitors and carbonic anhydrase inhibitors are useful for treatment of corneal graft rejection, ophthalmic or ocular neovascularization, retinal neovascularization including that following injury or infection, diabetic retinopathy, macular degeneration, retrolental fibroplasia and neovascular glaucoma. International Patent Publication No. WO 00/32189, which is incorporated herein by reference, describes orally deliverable compositions of celecoxib having utility in treatment of ophthalmic diseases such as retinitis, conjunctivitis, retinopathies, uveitis and ophthalmic or ocular photophobia, and of acute injury to eye tissue. It is describes that the subject orally deliverable compositions are useful for treatment of corneal graft rejection, ophthalmic or ocular neovascularization, retinal neovascularization including that following injury or infection, diabetic retinopathy, macular degeneration, retrolental fibroplasia and neovascular glaucoma.

One embodiment of the present invention provides a method of treatment of ophthalmologic disorders, diseases or conditions in which carbonic anhydrase is implicated or involved in metabolic pathways that influence the disorder, disease or condition comprising therapeutically effective amounts of Compound I, II, III, IV, V, VI or VII in combination with other glaucoma drugs whether or not the agents are administered orally, topically to the eye or other method of delivery, the glaucoma drugs including, but not limited to, acetazolamide; osmotic diuretics; pilocarpine; beta blockers.

Further, the present invention includes the treatment of ophthalmological diseases or disorders comprising the administration of therapeutically effective amounts of Compounds I, II, III, IV, V, VI or VII with one or more intraophthalmic or ocular pressure-reducing drugs including, without limitation latanoprost, travoprost, bimatoprost, or unoprostol.

Any drug having utility in a topical ophthalmic application can be used in co-therapy, co-administration or co-formulation with Compound I, II, III, IV, V, VI or VII in methods of treatment of ophthalmological diseases or disorders in which carbonic anhydrase is implicated or involved in metabolic pathways that influence the diseases or conditions. Such drugs include without limitation demulcents; antibiotics, antivirals and other anti-infectives; steroids, NSAIDs and other anti-inflammatory agents; acetylcholine blocking agents; adrenergic agonists, beta-adrenergic blocking agents and other antiglaucoma agents; antihypertensives; antihistamines; anticataract agents; and topical and regional anesthetics. Illustrative specific drugs include acebutolol, aceclidine, acetylsalicylic acid (aspirin), N⁴ acetylsulfisoxazole, alclofenac, alprenolol, amfenac, amiloride, aminocaproic acid, p-aminoclonidine, aminozolamide, anisindione, apafant, atenolol, bacitracin, benoxaprofen, benoxinate, benzofenac, bepafant, betamethasone, betaxolol, bethanechol, brimonidine, bromfenac, bromhexine, bucloxic acid, bupivacaine, butibufen, carbachol, carprofen, cephalexin, chloramphenicol, chlordiazepoxide, chlorprocaine, chlorpropamide, chlortetracycline, cicloprofen, cinmetacin, ciprofloxacin, clidanac, clindamycin, clonidine, clonixin, clopirac, cocaine, cromolyn, cyclopentolate, cyproheptadine, demecarium, dexamethasone, dibucaine, diclofenac, diflusinal, dipivefrin, dorzolamide, enoxacin, eperezolid, epinephrine, erythromycin, eserine, estradiol, ethacrynic acid, etidocaine, etodolac, fenbufen, fenclofenac, fenclorac, fenoprofen, fentiazac, flufenamic acid, flufenisal, flunoxaprofen, fluorocinolone, fluorometholone, flurbiprofen and esters thereof, fluticasone propionate, furaprofen, furobufen, furofenac, furosemide, gancyclovir, gentamycin, gramicidin, hexylcaine, homatropine, hydrocortisone, ibufenac, ibuprofen and esters thereof, idoxuridine, indomethacin, indoprofen, interferons, isobutylmethylxanthine, isofluorophate, isoproterenol, isoxepac, ketoprofen, ketorolac, labetolol, lactorolac, levo-bunolol, lidocaine, linezolid, lonazolac, loteprednol, meclofenamate, medrysone, mefenamic acid, mepivacaine, metaproterenol, methanamine, methylprednisolone, metiazinic, metoprolol, metronidazole, minopafant, miroprofen, modipafant, nabumetome, nadolol, namoxyrate, naphazoline, naproxen and esters thereof, neomycin, nepafenac, nitroglycerin, norepinephrine, norfloxacin, nupafant, olfloxacin, olopatadine, oxaprozin, oxepinac, oxyphenbutazone, oxyprenolol, oxytetracycline, penicillins, perfloxacin, phenacetin, phenazopyridine, pheniramine, phenylbutazone, phenylephrine, phenylpropanolamine, phospholine, pilocarpine, pindolol, pirazolac, piroxicam, pirprofen, polymyxin, polymyxin B, prednisolone, prilocaine, probenecid, procaine, proparacaine, protizinic acid, rimexolone, salbutamol, scopolamine, sotalol, sulfacetamide, sulfanilic acid, sulindac, suprofen, tenoxicam, terbutaline, tetracaine, tetracycline, theophyllamine, timolol, tobramycin, tolmetin, triamcinolone, trimethoprim, trospectomycin, vancomycin, vidarabine, vitamin A, warfarin, zomepirac and pharmaceutically acceptable salts thereof.

The invention provides that Compound V (celecoxib) and Compound VII (valdecoxib) can be administered alone to a subject having a disease or condition in which carbonic anhydrase is a factor and in which inflammation is present.

In another embodiment of the present invention, carbonic anydrase inhibitors, preferably, Compounds I, II, III, IV, V, VI or VII, are combined with antineoplastic drugs or agents, anticancer drugs or agents or antiangiogenic drugs or agents in methods of treatment and prevent of diseases in which carbonic anhydrase inhibitors combined with antineoplastic drugs or agents, anticancer drugs or agents or antiangiogenic or antineoplastic agents are effective

The Compounds I, II, III, IV, V, VI or VII are combined with antineoplastic agents which include antimetabolite agents, alkylating agents, antibiotic-type agents, hormonal anticancer agents, immunological agents, interferon-type agents, and a category of miscellaneous ounantineoplastic agents to treat neoplastic diseases or conditions in which carbonic anhydrase also is implicated. These neoplastic diseases and conditions include, but are not limited to, renal cancer, leukemia, non-small cell lung cancer, ovarian cancer, melanoma, colon, CNS, renal, prostate and breast cancer cell lines. Even more preferably the compounds I, II, III, IV, V, VI or VII re combined with pyrimidine analogs and, more preferably, the compounds are used in combinations with 5 fluorouracil (5-FU) and prodrugs of 5-FU such as 1-phthalidyl 5 fluorouracil (PH-FU) to enhance effectiveness of the I, II, III, IV, V, VI or VII.

As set out above, related compounds, for example compounds having the general structure of Compound A, which include a sulfonamide group and exhibit carbonic anhydrase, may be used in the methods of the present invention and are intended to be included within the scope of the appended claims. Therefore, the foregoing description and examples are intended to be illustrative of the methods of the present invention and should not be construed in a limiting sense.

What is claimed is:

1. A method of treating or preventing a carbonic anhydrase associated disorder in a subject in need of such treatment or prevention comprising the administration to the subject a carbonic anhydrase inhibitor treating- or preventing-effective amount of a cyclooxygenase-2 inhibitor, a pharmaceutically acceptable salt thereof or prodrug to treat or prevent the disorder.

2. The method of claim 1 wherein in the cyclooxygenase-2 inhibitors is a compound of the formula 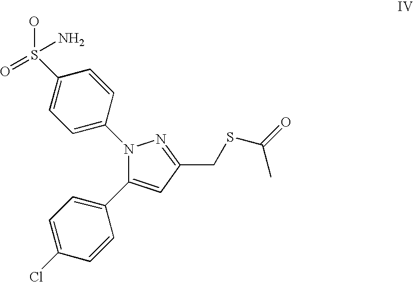
or a pharmaceutically acceptable salt thereof or prodrug.

3. The method of claim 1 wherein the cyclooxygenase-2 inhibitor is a selective cyclooxygenase inhibitor.

4. The method of claim 3 wherein the selective cyclooxygenase-2 inhibitor is a compound selected from the group consisting of 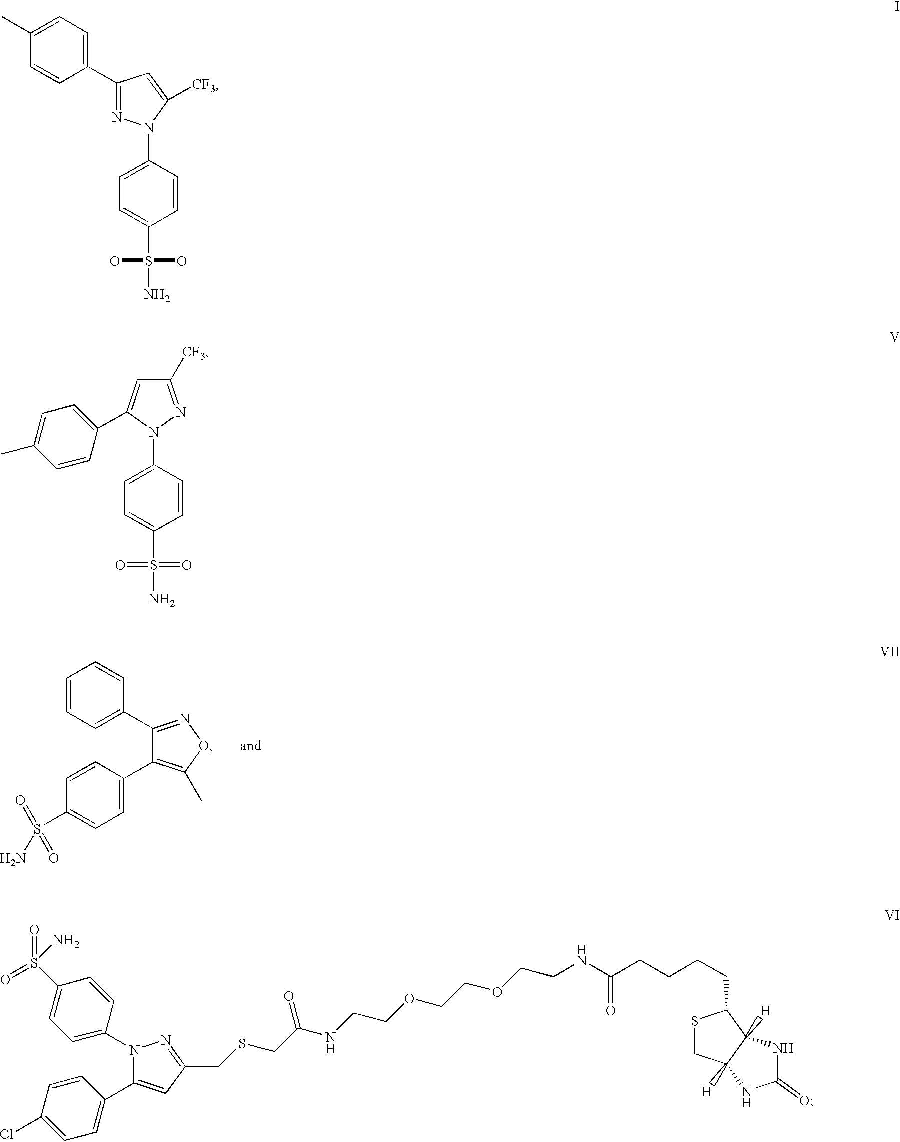
or pharmaceutically acceptable salt thereof or prodrug.

5. The method of claim 3 wherein the selective cyclooxygenase-2 is compound selected from the group of compounds consisting of the formulas 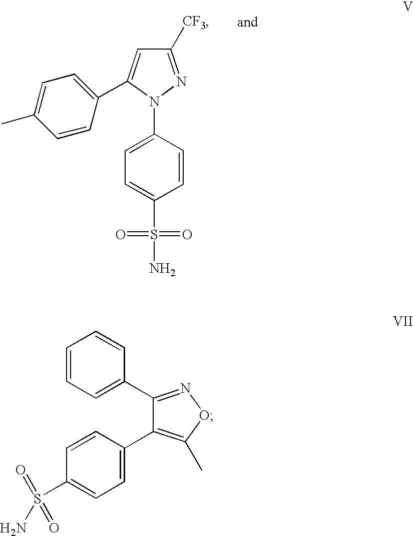
or pharmaceutically acceptable salt thereof or prodrug.

6. The method of claim 5 wherein the prodrug of compound formula 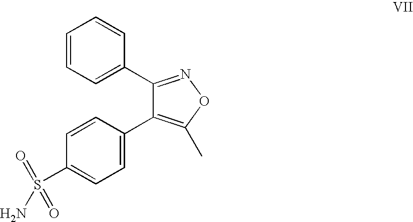
is compound formula

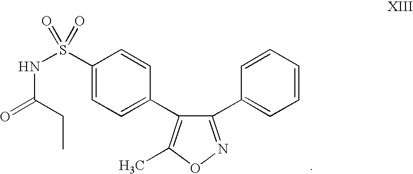

7. The method of claim 1 wherein the disorder is selected from a group of disorders comprising edema associated with congestive heart failure, drug-induced edema, open-angle glaucoma, secondary glaucoma, acute angle closure glaucoma, epilepsy, acute mountain sickness, familial periodic paralysis, metabolic alkalosis, optic neuropathy, pseudomotor cerebri, cystoid macular edema and cystoid macular edema.

8. The method of claim I wherein the disorder is a neoplastic disorder.

9. The method of claim 8 wherein the neoplastic disorder is selected from the group of neoplastic disorders comprising renal cancer, leukemia, lung cancer, ovarian cancer, melanoma, colon cancer, cancer of the central nervous system, prostate cancer and breast cancer.

10. A method of treating or preventing a carbonic anhydrase associated disorder in a subject in need of such treatment or prevention comprising the administration to the subject a carbonic anhydrase associated disorder treating- or preventing-effective amount of a cyclooxygenase-2 inhibitor compound having a sulfonamide structure thereon, a pharmaceutically acceptable salt thereof or prodrug to treat or prevent the disorder.

11. The method of claim 10 wherein the cyclooxygenase-2 inhibitor compound having a sulfonamide structure thereon is selected from the group consisting of 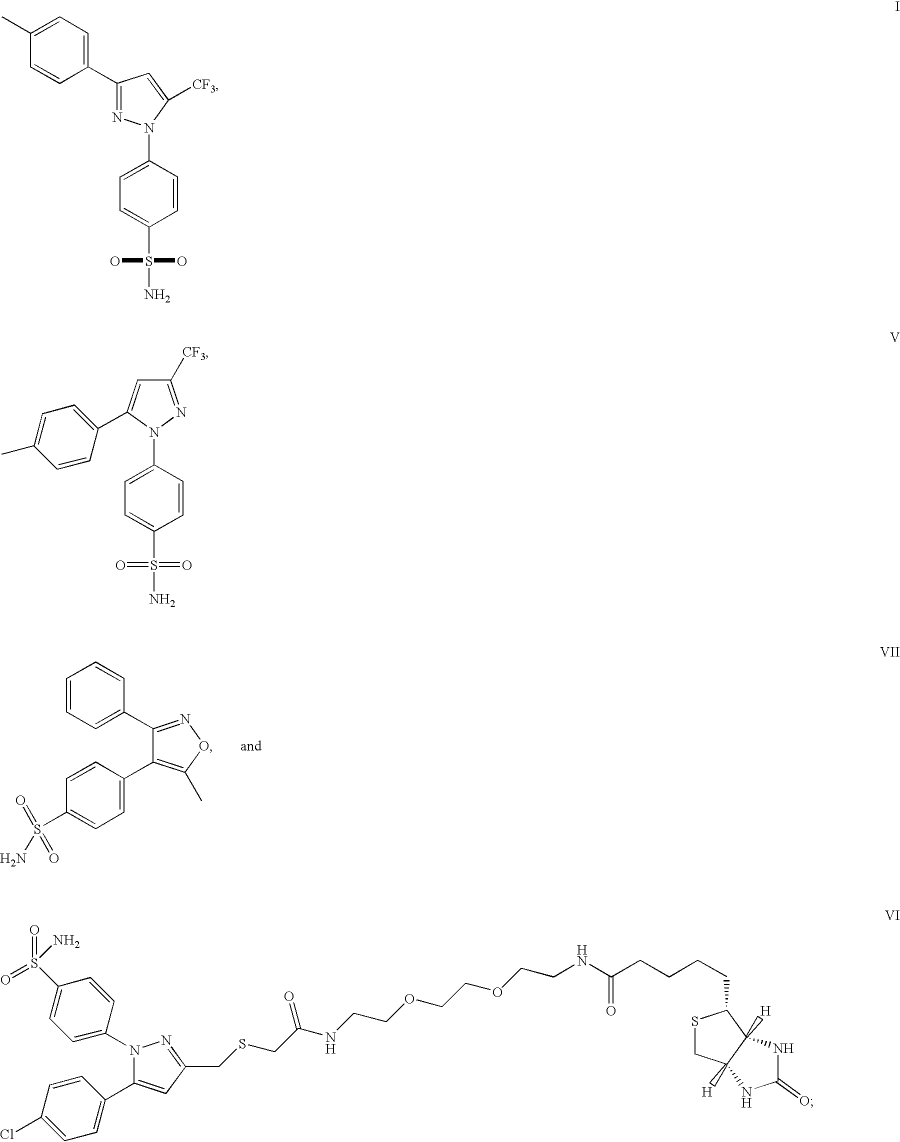
or pharmaceutically acceptable salt thereof or prodrug.

12. The method of claim 10 wherein the cyclooxygenase-2 inhibitor compound having a sulfonamide structure thereon is selected from the group consisting of 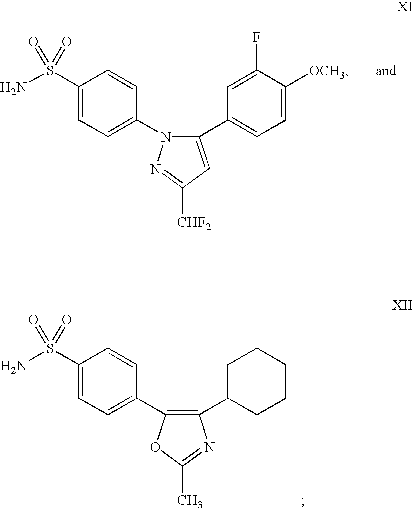
or pharmaceutically acceptable salt thereof or prodrug.

13. The method of claim 10 wherein the carbonic anhydrase associated disorder is a neoplastic disorder selected from the group of neoplastic disorders consisting of renal cancer, leukemia, lung cancer, ovarian cancer, melanoma, colon cancer, cancer of the central nervous system, prostate cancer and breast cancer.

14. The method of claim 10 wherein the carbonic anhydrase associated disorder is an ophthalmic disorder selected from the group of ophthalmic disorder consisting of open angle glaucoma, acute angle closure glaucoma, optic neuropathy, and cystoid macular edema.

15. A method of treatment of a neoplastic disorder or disease comprising the administration of neoplastic disorder- or disease treatment-amount of a carbonic anhydrase inhibitor compound selected from the group of compounds of the formula 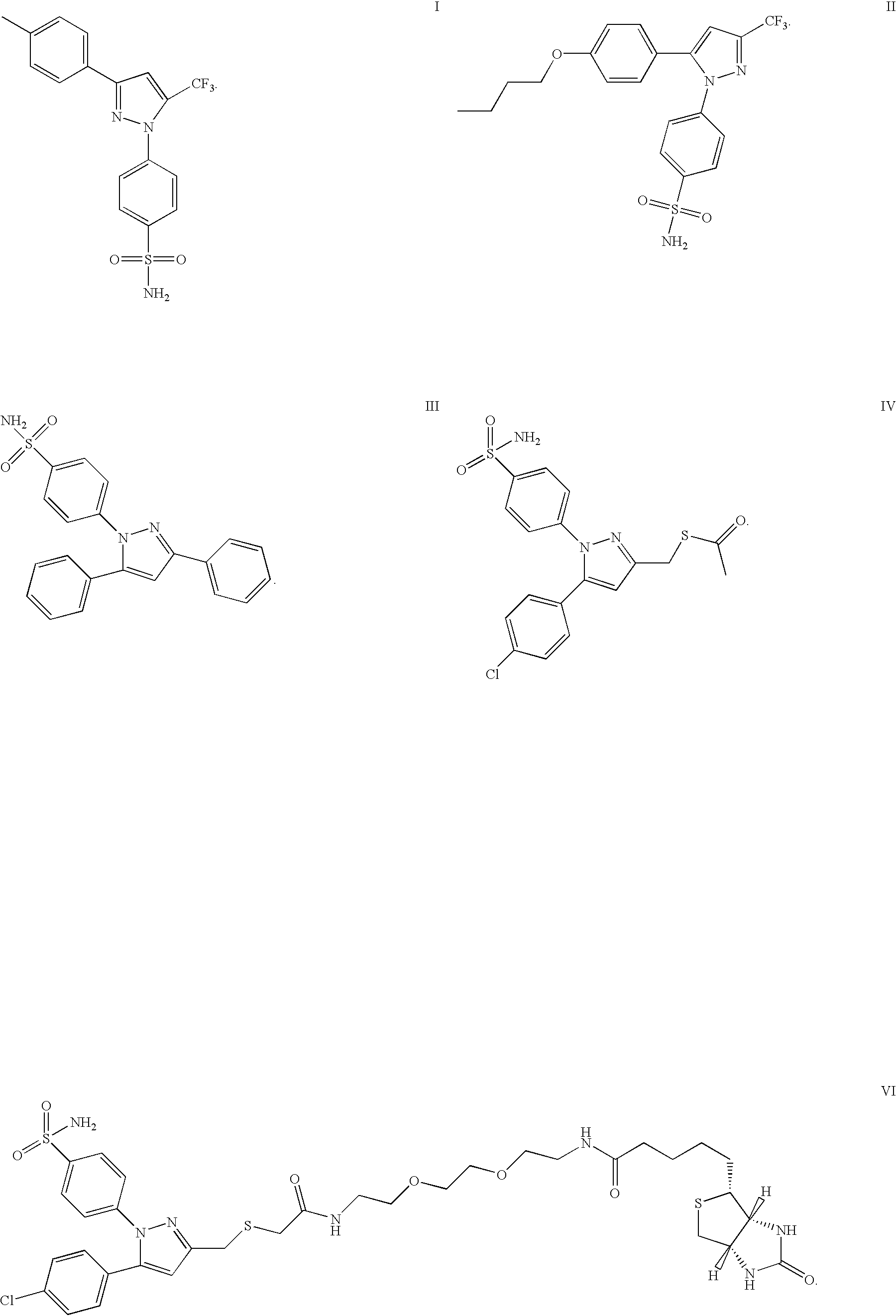
a pharmaceutically acceptable salt thereof or prodrug.

16. The method of claim 15 wherein the neoplastic disorder or disease is selected from the group of neoplastic disorders comprising renal cancer, leukemia, lung cancer, ovarian cancer, melanoma, colon cancer, cancer of the central nervous system, prostate cancer, and breast cancer.

17. A method for treating or preventing a neoplastic disease or disorder in a subject in need of such treatment or prevention, comprising administering to the subject an amount of an antineoplastic agent and an amount of a carbonic anhydrase inhibitor compound, said carbonic anhydrase inhibitor compound selected from the group of carbonic anhydrase inhibitor compounds consisting of the compounds of formula 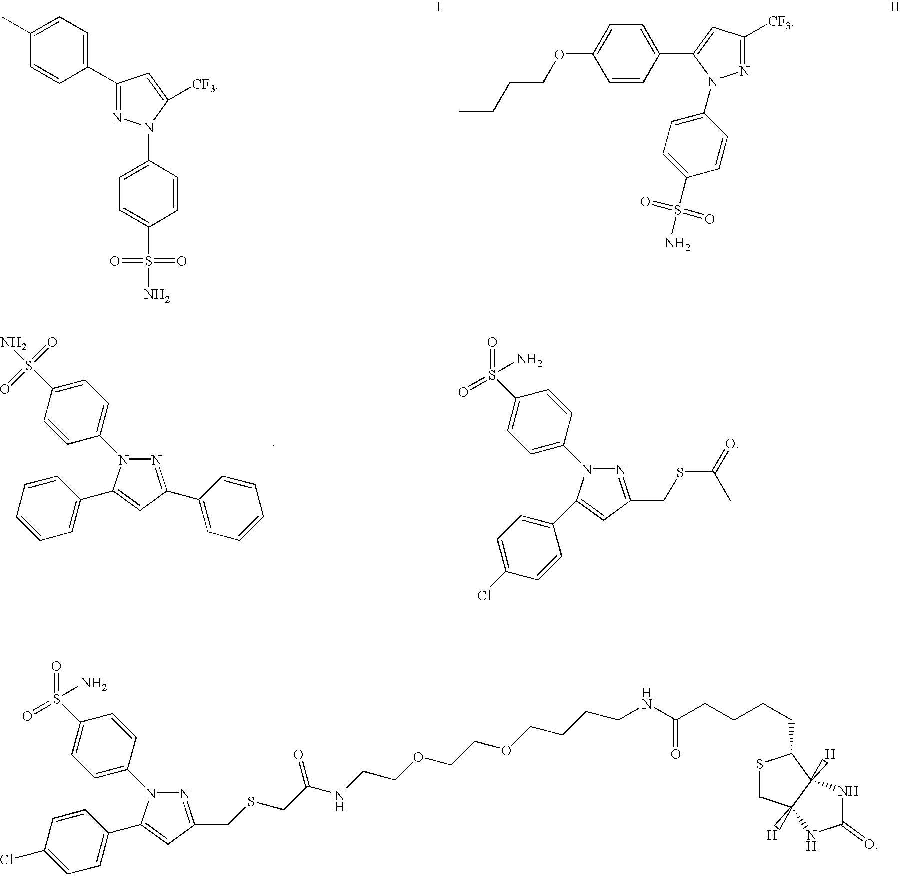
a pharmaceutically acceptable salt thereof or prodrug; and
wherein the amount of the antineoplastic agent and the amount of the carbonic anhydrase inhibitor compound together comprise a neoplastic disorder treating- or preventing-amount of the antineoplastic agent and the carbonic anhydrase inhibitor compound.

18. The method of claim 17 wherein said antineoplastic agent is selected from the group consisting of an antimetabolite agent, an alkylating agent, an antibiotic-type agent, a hormonal anticancer agent, an immunological agent, and an interferon-type agent.

19. The method of claim 17 wherein the antineoplastic agent is 1-phthalidyl 5-fluorouracil.
